(19) United States
(12) Patent Application Publication
(10) Pub. No.: US 2016/0310316 A1
(43) Pub. Date: Oct. 27, 2016

(54) Devices and Methods for Hypothermia Amelioration in Non-Human Mammals

(71) Applicant: AVAcore Technologies, Inc., Ann Arbor, MI (US)

(72) Inventor: Charles A. Hixson, JR., Ann Arbor, MI (US)

(21) Appl. No.: 15/140,352

(22) Filed: Apr. 27, 2016

Related U.S. Application Data
(60) Provisional application No. 62/153,260, filed on Apr. 27, 2015.

(51) Int. Cl. A61F 7/02

(52) U.S. Cl. CPC A61F 7/02; A61F 2007/0029

(57) ABSTRACT

Devices and methods for ameliorating hypothermia or risk of occurrence thereof in non-human mammals are provided. Embodiments of the subject devices include a limb enclosure unit and a liquid flow path associated with the enclosure unit. In some versions, the liquid flow path is configured to bring a warm liquid in heat transfer relationship with a limb of a small non-human mammal present in the enclosure unit. Methods for using such devices are also provided and in some versions include moving a warm liquid through the flow path in a manner sufficient to ameliorate hypothermia or the risk of occurrence thereof in the small non-human mammal.

### CROSS-REFERENCE TO RELATED APPLICATIONS

Pursuant to 35 U.S.C. §119 (e), this application claims priority to the filing date of U.S. Provisional Patent Application Ser. No. 62/153,260 filed Apr. 27, 2015; the disclosure of which application is herein incorporated by reference.

### INTRODUCTION

Hypothermia is a condition in which body temperature becomes too low for normal functioning, such as normal metabolic functioning. Hypothermia may occur in mammals including non-human mammals such as dogs and cats. Hypothermia is more common in animals that are wet, small, have short hair or lack shelter in a cold environment. Signs of hypothermia in a non-human mammal may include shivering and paleness of skin. Lethargy, listlessness and frostbite may also occur. If sustained, a low body temperature associated with hypothermia may lead to a variety of severe health complications including coma and even death resulting from, for example, heart failure.

### SUMMARY

Devices and methods for ameliorating hypothermia or risk of occurrence thereof in non-human mammals are provided. Embodiments of the subject devices include a limb enclosure unit and a liquid flow path associated with the enclosure unit. In some versions, the liquid flow path is configured to bring a warm liquid in heat transfer relationship with a limb of a small non-human mammal present in the enclosure unit. Methods for using such devices are also provided and in some versions include moving a warm liquid through the flow path in a manner sufficient to ameliorate hypothermia or the risk of occurrence thereof in the small non-human mammal.

### BRIEF DESCRIPTION OF THE FIGURES

FIG. 1 provides a partial cross-sectional view of a device according to one embodiment of the subject disclosure.

FIG. 2 provides a top view of a device according to one embodiment of the subject disclosure.

FIG. 3 provides a view of several of the devices employed on a small-non human mammal undergoing a surgical procedure.

FIG. 4 provides a partial cross-sectional view of a device according to an embodiment of the subject disclosure.

### DETAILED DESCRIPTION

Devices and methods for ameliorating hypothermia or risk of occurrence thereof in non-human mammals are provided. Embodiments of the subject devices include a limb enclosure unit and a liquid flow path associated with the enclosure unit. In some versions, the liquid flow path is configured to bring a warm liquid in heat transfer relationship with a limb of a small non-human mammal present in the enclosure unit. Methods for using such devices are also provided and in some versions include moving a warm liquid through the flow path in a manner sufficient to ameliorate hypothermia or the risk of occurrence thereof in the small non-human mammal.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges may be presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing the subject invention, subject devices for use in practicing the subject methods will be discussed in greater detail, followed by a review of methods and particular applications.

### Devices

Aspects of the subject disclosure include devices for hypothermia amelioration. As used herein, the terms “amelioration” and “ameliorate” refer to improving and/or preventing a condition, such as hypothermia, or the risk of a condition, in the body of a subject. By “improving a condition”, is meant adjusting one or more characteristics associated with the condition, e.g., body temperature, such that the one or more characteristics are closer to or identical to what they would be in a normal, healthy subject. In some versions, amelioration may include adjusting, e.g., increasing and/or decreasing, one or more characteristics associated with a condition, e.g., body temperature, and/or maintaining one or more of such characteristics. In some aspects, amelioration may include minimizing a risk of an occurrence of a condition, such as hypothermia in a subject. Furthermore, the term “hypothermia”, as used herein, refers to subnormal temperature of the body of a subject. In some versions, hypothermia includes a reduction in the rate of one or more metabolic processes in the body of a subject.

More specifically, the subject disclosure provides devices for ameliorating surgical procedure associated hypothermia or risk of occurrence thereof in a subject, such as a non-human mammal. Surgical procedure associated hypothermia is hypothermia associated with, such as induced prior to and/or during a surgical procedure, or caused by one or more conditions related to a surgical procedure, e.g., anesthesia administration and/or trauma and/or bleeding. As used herein, the phrase “surgical procedure” refers to a procedure, e.g., a medical procedure, involving at least one incision in the body of a subject and/or performed using one or more instruments, e.g., surgical instruments. A surgical procedure may be carried out through a body cavity and/or through the skin of a subject. Also, as used herein, the phrase “risk of occurrence” refers to the probability that an occurrence, such as a condition, such as hypothermia, will take place. As such, ameliorating a risk of occurrence refers to reducing the probability that a condition, such as hypothermia, will occur in a subject.

A normal core body temperature, also referred to herein as body temperature, of a dog may range, for example, from 37° C. to 40° C., such as 38° C. to 39.2° C., such as 38° C. to 38.5° C., inclusive. A normal body temperature of a cat may range, for example, from 37° C. to 40° C., such as 37.8° C. to 39° C., such as 38° C. to 39° C., inclusive. Hypothermia, such as surgical procedure associated hypothermia and/or hypothermia in a dog and/or cat, may include a body temperature that is 1° C. or more, 5° C. or more, 10° C. or more, or 15° C. or more below normal body temperature. As such, hypothermia, such as hypothermia in a dog and/or cat, may include a body temperature below 37° C., such as below 35° C., such as below 32° C., such as below 28° C. Hypothermia, such as hypothermia in a dog and/or cat, may include a body temperature in a range, for example, of 1° C. to 37° C., such as 1° C. to 35° C., such as 1° C. to 28° C., or 28 to 32° C., or 32 to 35° C.

By “body core” and “core body” is meant the internal region of the mammal, as opposed to the surface of the mammal. The magnitude of core body thermal energy infusion accomplished during practice of the subject methods may vary, and is sufficient to provide for the desired outcome, e.g. an elevation in and/or maintenance of core body temperature, and/or amelioration of hypothermia or risk thereof. In certain embodiments, the magnitude of heat infusion is 0.5 Kcal/min or less, 1.0 Kcal/min or less, or 1.5 Kcal/min or less, where the magnitude may be 50 Kcal/min or greater, but sometimes is 30 Kcal/min or less, such as 20 Kcal/min or less. The period of time that the heat is infused to the body may range, for example, from 1 min to 24 hrs, such as from 2 min to 20 min, or from 2 min to 5 min, or for the duration or substantial duration of a surgical procedure.

The subject disclosure provides devices and methods for ameliorating hypothermia or risk of occurrence thereof in a non-human mammal, i.e., a mammal which is not a human. The terms “mammal” and “mammals” are used broadly herein to describe organisms which are within the class mammalia, including the orders carnivore (e.g., dogs and cats), rodentia (e.g., mice, guinea pigs, and rats), and non-human primates (e.g., chimpanzees, and monkeys). Mammals may be subjects, such as canine and/or feline subjects. The terms “mammal” and “mammals” may include both genders and at any stage of development (i.e., fetal, neonates, infant, juvenile, adolescent, adult), where in certain embodiments the mammal is a juvenile, adolescent or adult. Also, non-human mammals according to the subject disclosure, may be small non-human mammals. Small non-human mammals are mammals which are generally smaller, e.g., have a smaller weight and/or body volume, than an average adult human and include dogs, cats, mice, guinea pigs, and rats. Small non-human mammals may have a weight of 80 kg or less, such as 40 kg or less, such as 20 kg or less. Non-human mammals may include, for example, dogs, cats, monkeys, rats, rabbits, chipmunks, marmots, ferrets, guinea pigs, mice, hamsters, and the like.

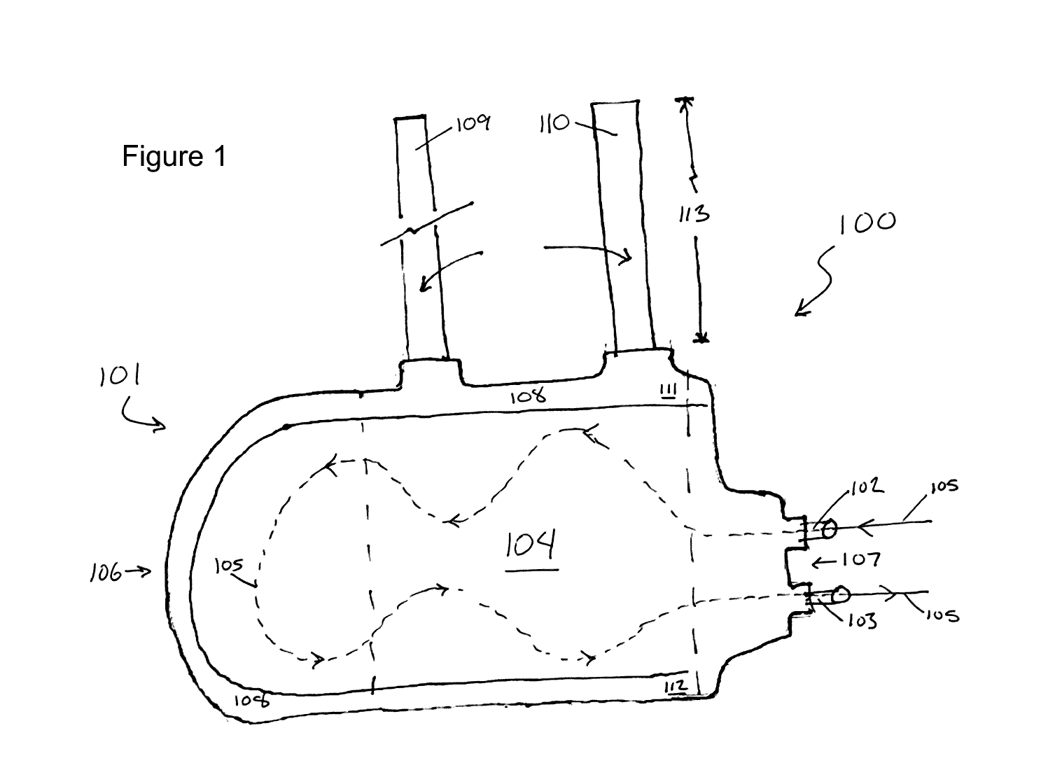

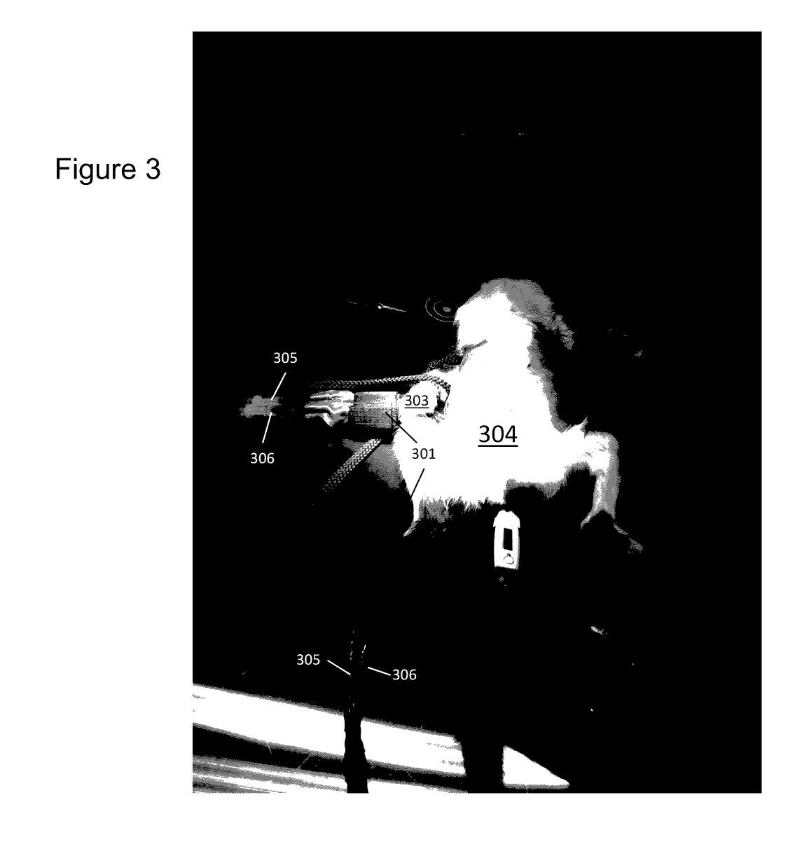

One embodiment of a device for ameliorating surgical procedure associated hypothermia or risk of occurrence thereof for use in practicing the subject methods is provided in FIG. 1. In various embodiments, a device 100 includes a limb enclosure unit 101. As is illustrated, for example in FIG. 3, one or more limb enclosure units 301 may, in some versions, be configured to enclose a portion, such as at least a substantial portion, of one or more limbs 303 of a small non-human mammal 304 undergoing a surgical procedure.

Embodiments of the devices also include a liquid flow path associated with the enclosure unit 101. A liquid flow path may include, for example, a liquid inlet 102, a liquid outlet 103 and/or a chamber 104, such as a chamber 104 in operative, e.g., fluidic, communication with the liquid inlet 102 and/or the liquid outlet 103. As used herein, the phrase “operative communication” refers to being associated with, e.g., connected, in a specific way that allows the disclosed devices to operate and/or methods to be carried out effectively in the manner described herein. Operative communication may include fluidic communication, in which one or more fluids are able to flow between a first aspect and a second aspect in fluidic communication with the first aspect. Operative communication may also include being connected in a manner allowing electric power and/or thermal energy, e.g., heat, to be transmitted, and/or in a manner physically coupling e.g., releasably or fixedly coupling, one aspect to another. In addition, a liquid flow path may be configured to bring a liquid, e.g., a warm liquid having a flow shown schematically in FIG. 1 as element 105, in heat transfer relationship with a limb of a non-human mammal, e.g., a small non-human mammal, present in the enclosure unit 101.

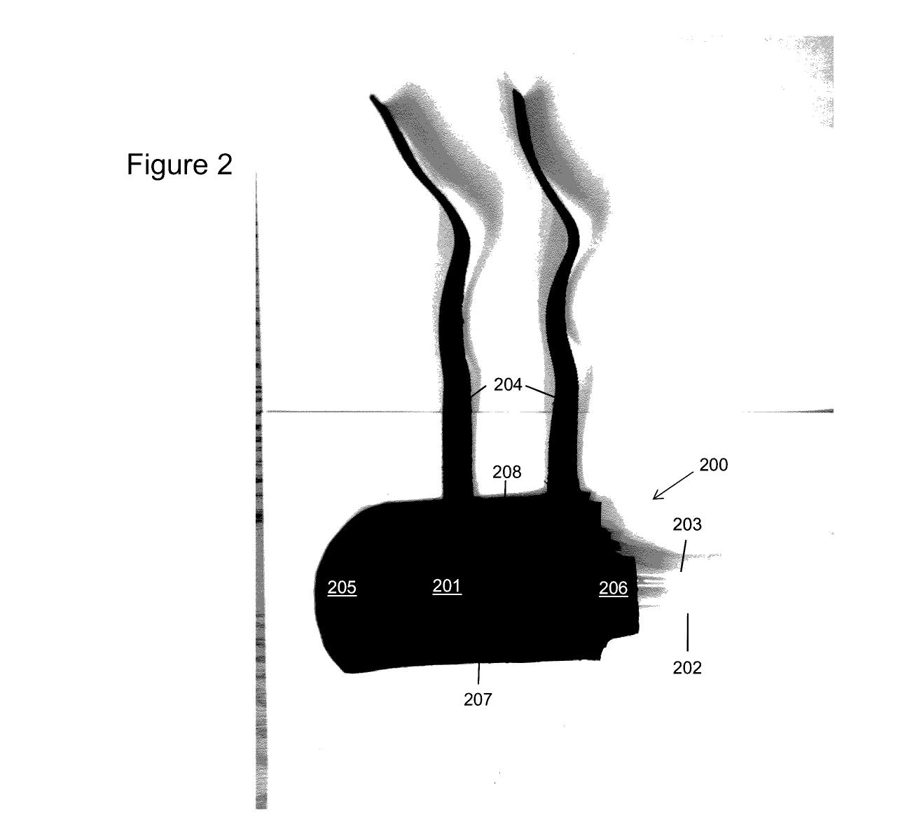

FIG. 2 provides a device 200 embodiment including an enclosure unit 201 having a first end 205 and a second end 206 opposite the first end, a liquid inlet 202, and a liquid outlet 203. As shown, first end 205 is closed. However, in some embodiments, the first end 205 may not be entirely closed, i.e., it may be open. Also provided in the depicted embodiment are limb securing elements 204. The device 200 also has a bottom edge 207 and a top edge 208 opposite the bottom edge and to which the limb securing elements 204 are attached, e.g., fixedly attached. FIG. 3 also illustrates an embodiment including liquid inlets 305, and liquid outlets 306. Additionally, as shown in FIG. 3, a thermometer 307 may be used to monitor the body temperature of a non-human mammal during a surgical procedure.

As noted above, in some embodiments, a limb enclosure unit is configured to enclose a limb of a non-human mammal, or a portion thereof. By “enclose” is meant to contain therein. As such, the term “enclose” may include containing a portion of a limb between a first portion of the limb enclosure unit and a second portion of the limb enclosure unit, e.g., a portion opposite the first. Enclosing a portion of a limb may also include surrounding or contacting the limb on two or more sides, such as four sides or five sides. A portion of a limb of a mammal enclosed in a limb enclosure unit may be a portion of a mammal which lies partially or substantially within, such as between two or more portions, e.g., opposite interior portions of, or encapsulated within, a limb enclosure unit. An enclosed portion of a mammal may be a portion of a mammal that is contained within or surrounded by a limb enclosure unit.

According to some embodiments, a limb enclosure unit is configured to enclose at least a substantial portion of a limb of a non-human mammal. As used herein, the terms “substantial” and “substantially” refer to a great or significant extent, such as almost fully or almost entirely. In various embodiments, enclosure units are configured to enclose 10% or more, such as 50% or more, such as 90% or more of the surface area, e.g., the external surface area, of a limb on a small non-human mammal.

An appendage, limb or a portion thereof which may be enclosed in a limb enclosure unit may be, for example, a leg, or a portion thereof. A limb or a portion thereof which may be enclosed in a limb enclosure unit may be a portion including and between the end of the limb, e.g., the end of the longest digit, and a shoulder, and/or elbow, and/or forearm, and/or carpus, and/or wrist, and/or metacarpus, and/or pastern, and/or forefoot. A limb or a portion thereof which may be enclosed in a limb enclosure unit may include one or more digits, e.g., toes, forefoot, declaw, pastern carpal, i.e., wrist, forearm, shoulder, or paw, or any combination or portion, thereof.

In some aspects, the enclosure units disclosed herein are configured as a boot, such as a boot configured for wearing by a non-human mammal. As such, an enclosure unit may be a container, e.g., a pouch, and/or sheath having a single opening therein for receiving a portion of a non-human animal, such as a limb, or a portion thereof. An enclosure unit, in various aspects, may include a pocket into which a limb of a non-human mammal may be inserted. An enclosure unit may fit snugly over an end of a limb of a mammal. An enclosure unit may also be or include a flexible sheet which can be flexed, e.g., rolled, into a container, e.g., a cylinder, for receiving a limb. By “flexible”, as used herein is meant pliable or capable of being bent or flexed repeatedly (e.g., bent or flexed with a force exerted by a human hand or other body part) without damage (e.g., physical deterioration).

In some embodiments, enclosure units include one or more walls, such as a first wall and a second wall opposite the first wall. Each wall may include a layer of material and may be attached, e.g., fixedly and/or sealably attached, to one or more other wall. A layer of one or more materials of a limb enclosure unit may also include a first wall on a first side of the layer and a second wall on a second side of the layer which is opposite the first side.

The walls of the subject enclosure units, e.g., a first wall and a second wall, may be configured to contain a limb or a portion thereof between them. In some versions, the subject walls, e.g., a first wall and a second wall, are separated by a solid layer of material. In some aspects, one or more walls, e.g., two walls, three walls, four walls, etc., of an enclosure unit are limb associating walls. A limb associating wall is a wall which is positioned within the device such that it, or a surface thereof, associates with, e.g., contacts, a limb when a limb is contained within the enclosure unit. A limb associating wall may have a first surface, e.g., a limb associating surface, which contacts a limb when a limb is within a limb enclosure unit, and a second surface opposite the first surface. In various embodiments, enclosure units include an enclosure unit wall that is opposite a limb associating wall of the enclosure unit. An enclosure unit wall that is opposite a limb associating wall of the enclosure unit may have a surface area which may be associated with, for example, a chamber of a liquid flow path.

A limb associating wall may include one or more surfaces, e.g., a heat exchange surface, for contacting and/or transferring heat with a portion of a non-human mammal e.g., a limb. Such heat exchange surfaces, in some embodiments, are sized and shaped to, together with other portions, e.g. walls of an enclosure device, define a smooth, e.g., lacking any distinct edges, interior cavity of a device as described herein.

Aspects of the subject devices, such as limb enclosure units and liquid flow paths or components thereof may include or be composed of one or more flexible materials. A flexible material may be a material that remains able to perform intended function (e.g., repeatedly flexing) by remaining pliable for at least the expected lifetime or useful lifetime of the aspect which the material is included in. Aspects of the subject devices, such as limb enclosure units and liquid flow paths or components thereof may include or be composed of one or more biocompatible materials. The phrase “biocompatible materials” are materials that can be placed on or in living tissue for an extended period of time, such as for a period of 1 hour or more, such as 2 hours or more, such as 10 hours or more, such as 1 day or more, such as 2 days or more, such as 1 week or more, 4 weeks or more, 6 months or more, or 1 year or more, e.g., 5 years or more, up to and including the remaining lifetime or expected remaining lifetime of the subject or more, and not cause a significant adverse (e.g., detrimental to health) reaction (e.g., an immune response) in the tissue or the associated organism.

As is shown, for example, in FIG. 1, in some aspects, an enclosure unit 101 as disclosed herein includes a first end, e.g., a rounded end, and a second end 107 opposite the first end, e.g., an end at which a liquid inlet 102 and/or liquid outlet 103 of the liquid flow path are positioned. An enclosure unit may also include a sealing area 108, e.g., a weld area, extending around its periphery which is configured to retain a portion of a limb of a mammal within the enclosure unit. A sealing area 108, e.g., a weld area, may also, in some versions help define the chamber 104 of the liquid flow path. In some versions, the enclosure unit 101 is configured to receive a limb or a portion thereof into an opening at the second end 107 of the device. In some versions, such an opening is an opening defined between a first end 111 of a sealing area 108 and second end 112 of a sealing area 108. As such, a limb or a portion thereof, e.g., an end of a limb, may be inserted into a sealing enclosure 101 from a second end 107 toward a first end 106.

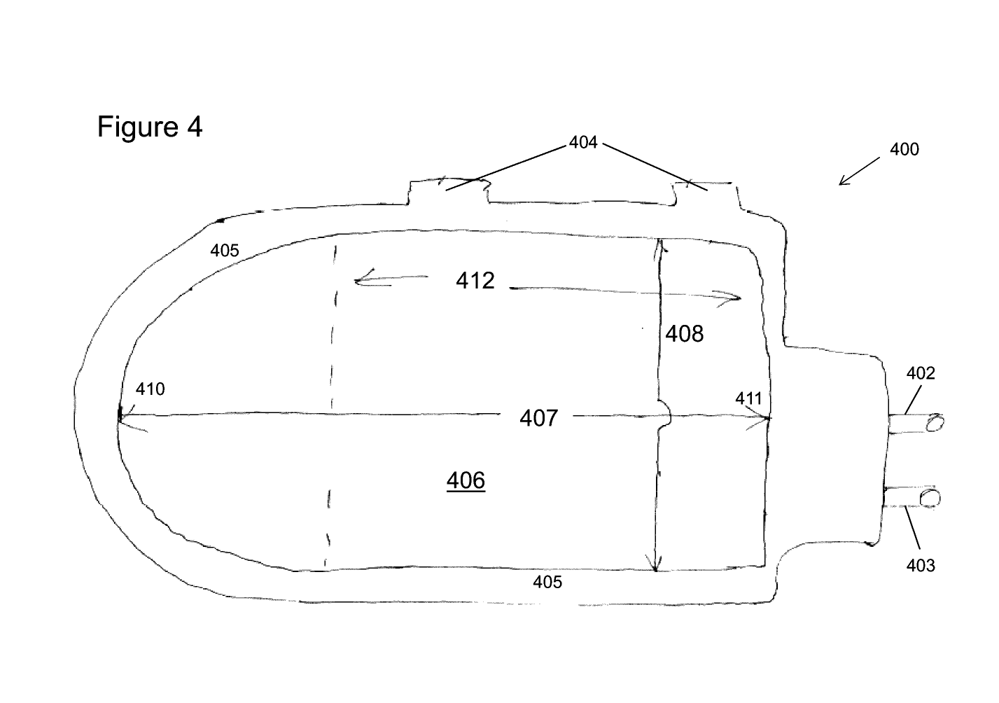

FIG. 4 shows an embodiment of a device 400 for ameliorating hypothermia or a risk thereof in a non-human mammal. The device 400 includes an enclosure unit 401 and a liquid flow path having a liquid inlet 402 and a liquid outlet 403. The device 400 also includes attachment protrusions 404 for attaching to limb securing elements. In addition, the device 400 includes a periphery 405, e.g., a sealed periphery, defining a cavity 406 configured for receiving a portion of a limb of a non-human mammal therein. The cavity 406 has dimensions including a length 407, which may be 15.88 cm, width 408, which may be 8.26 cm and height. In some embodiments, the length 407 may range, for example, from 5 cm to 30 cm, such as 10 cm to 25 cm, such as 10 cm to 15 cm. The width dimension and the height dimension may be the same length or the width dimension may be longer than the height dimension. In some embodiments, the width 408 and/or height may range, for example, from 1 cm to 20 cm, such as 1 cm to 10 cm, such as 1 cm to 5 cm. The cavity 406 may have a first 410 end and a second end 411 opposite the first end and/or proximate the liquid inlet 402 and/or the liquid outlet 403. The device 400 may also include a turbulence inducer 409 spaced from a second end 411 of a cavity 406 by a distance 412, which may be 10.16 cm. In some embodiments, such a distance may range, for example, from 1 cm to 20 cm, such as 1 cm to 15 cm, such as 5 cm to 10 cm. Such a device 400 may also include a second turbulence inducer at the location of width 408 as shown in FIG. 4.

A limb of a non-human mammal or a portion thereof may be placed against the enclosure unit 101 and/or at least a portion of the enclosure unit 101 wrapped and/or secured around the portion of the limb of the mammal. Accordingly, in some embodiments, the subject devices include one or more limb securing elements, such as a first limb securing element 109 and second limb securing element 110, each having a length 113. Such limb securing elements may be configured to secure a limb of a mammal within a limb enclosure unit 101. Limb securing elements may be straps, such as adjustable straps, such as length-adjustable straps, and may each be attached to an edge of a limb enclosure unit at a first end of each securing element. Limb securing elements may be configured to tighten and/or loosen an enclosure unit or a portion thereof around a portion of a mammal so that, for example, the limb is securely fastened within the enclosure unit. Limb securing elements may be configured to adjustably shrink and/or enlarge an opening of an enclosure unit.

Limb securing elements may also include one or more attaching elements which are configured to attach, e.g., removably attach, a first portion of a limb securing element to a second portion of a limb securing element and/or another portion of a device, such as an enclosure unit. Attachment elements according to the subject disclosure may include, for example, snaps, buttons, hook-and-loop fasteners, reciprocating clamps, or any combinations thereof. Securing elements may also have one or more elastic portions and may be biased to return to their original shape.

In some versions, straps, e.g., adjustable straps, according to the subject disclosure are each associated with, e.g., adjacent to and/or helping to define, an opening of the enclosure unit. Each limb securing element may be rectangular and extend a length from the enclosure unit to a second end of each securing element. Such a length may be 21 cm or less, such as 20 cm or less, such as 10 cm or less, such as 5 cm or less. Each limb securing element may also have a width of 3 cm or less, such as 1.5 cm or less, such as 1 cm or less.

In some embodiments, the subject devices include a limb enclosure unit. The volume of the limb enclosure unit, or a portion thereof, such as a limb receiving pocket, may vary, ranging in some instances from 1 cm³ to 20 cm³, such as from 1 cm³ to 15 cm³, including from 5 cm³ to 10 cm³. Such a volume may be substantially the same volume as, or a slightly larger volume (e.g., greater than by 1 cm³ or less, 5 cm³ or less, or 10 cm³ or less) than the volume of a portion, e.g., limb, of a non-human mammal enclosed within the enclosure unit. Such a volume may be sized to fit, e.g., to encapsulate, 95% of small non-human mammal limb sizes and/or 95% of small non-human mammal limb sizes.

In some embodiments, the subject devices and/or the enclosure units thereof are configured to be portable. For example, the subject devices and/or the enclosure units and/or negative pressure elements thereof may be sized and shaped to be easily moved from one location to another by an amount of force capable of being exerted by an average child and/or adult human arm and/or hand. In certain aspects, the subject devices, e.g., devices including the enclosure units and/or flow paths described herein, have a mass ranging, for example, from 10 g to 100 g, or 100 g to 3000 g, from 140 g to 2900 g, from 500 g to 2000 g, or from 1300 g to 1400 g.

As noted above, various embodiments of limb enclosure units include one or more openings (e.g., two, three, four, five openings) configured to receive a portion of a small non-human mammal. Such an opening may separate the interior form the exterior of the negative pressure element and may be shaped as any convenient shape, e.g., a circle, oval, square, rectangle, triangle, or any combination thereof. An opening of a negative pressure element may also be sized to receive a portion of a non-human mammal therethrough, e.g., a hand and/or a foot. An opening of a limb enclosure unit may have, for example, an area of 1 cm² or less, 10 cm² or less, 25 cm² or less, 100 cm² or less, or 225 cm² or less. The opening of a limb enclosure unit may be the only single opening from the interior to the exterior of the element. If there are a plurality of openings on the unit, the openings may also be at opposite first and second ends of the unit.

As noted above, and illustrated in FIG. 1, the subject devices may include a liquid flow path associated with the enclosure unit 101. In other words, in some aspects, one or more portions of the liquid flow path are integral with and/or or attached to the enclosure unit, or one or more portions thereof. For example, a liquid flow path may include a chamber configured to have liquid flowed therethrough. Such a chamber may be integral with the enclosure unit or portions thereof. In some versions, such a chamber may be contained within the enclosure unit or portions thereof, e.g., a first surface, such as a top surface, and a second surface which is opposite the first surface, such as a bottom surface. A chamber may, in some embodiments, also be contained within the enclosure unit or portions thereof, such as a first material layer, e.g., a top material layer, and a second material layer opposite the first, e.g., a bottom material layer.

According to various embodiments, a liquid flow path may include and/or be defined by one or more containers, e.g., pouches, bags and/or conduits, such as tubes, or a series thereof which are operatively connected and/or in operative communication with one another. By “operatively connected” and “operatively connect”, as used herein, is meant connected in a specific way that allows the disclosed devices to operate and/or methods to be carried out effectively in the manner described herein. The containers of the flow path may have any shape and may be substantially cylindrical, cuboidal and/or tube-shaped. The liquid flow path or components thereof may be composed of any of the same materials as other portions of the device, e.g., a limb enclosure unit. Also, a liquid inlet, a liquid outlet, and/or a chamber may be flexible, e.g., composed of one or more flexible materials.

A liquid flow path may be configured such that liquid, e.g., a warm liquid, may flow therethrough, e.g., from a first portion, e.g., end, to a second portion, e.g., end, of a flow path. A flow path may have a first opening, e.g., configured for allowing fluid or a flow thereof into the flow path and a second opening configured for allowing fluid or a flow thereof out of the flow path.

In some versions, a liquid flow path includes a liquid inlet, a liquid outlet, and/or a chamber in fluid communication with the liquid inlet and/or the liquid outlet. In some versions, the liquid inlet and/or liquid outlet each include a conduit, e.g., tube, configured to receive a flow of fluid therethrough. A radius and/or diameter of a liquid inlet and/or liquid outlet may range, for example, from 1 mm to 30 mm, such as from 1 mm to 15 mm, such as from 5 mm to 10 mm, and may be 6.35 mm or 7.94 mm. A length, such as a length from a first end to a second end opposite the first end, of a liquid inlet and/or liquid outlet may range, for example, from 1 cm to 50 cm, such as from 1 cm to 25 cm, such as from 5 cm to 10 cm, and may be 7.62 cm.

The liquid inlet, a liquid outlet, and/or a chamber of the flow path may have any shape and may be substantially cylindrical, cuboidal and/or tube-shaped. In some aspects, a liquid inlet and a liquid outlet are both positioned at a first end of a chamber and/or a limb enclosure unit. In some aspects, a liquid inlet and a liquid outlet are positioned at opposite ends of a chamber and/or a limb enclosure unit. In some versions, a chamber of a liquid flow path includes an inner surface, e.g., a single, continuous inner surface, which is configured to contain liquid within the chamber. In some versions, a chamber is shaped as a conduit, e.g., a tubular conduit, which may be coiled, and directs a liquid flowing therethrough in a turbulent flow and/or a serpentine pattern. A chamber which is a conduit, e.g., a serpentine conduit, may extend around a portion of a limb and may direct fluid around at least a portion of the limb.

In some versions, a liquid flow path is sealed but for a first opening, such as a first opening in a liquid inlet, and a second opening, such as a second opening in a liquid outlet. In such a configuration a liquid may be configured to flow from the first opening to the second opening and/or from the second opening to the first opening.

Also, in some embodiments, a chamber of the flow path is attached to, e.g., fixedly attached to, and/or integrated with a limb enclosure unit. In some versions, a chamber of a flow path is adjacent to a limb enclosure unit, or a portion thereof. By “fixedly attached”, as used herein, is meant attached in an irreversible or substantially irreversible manner such that the aspect and/or the feature to which it is attached must be altered or damaged, e.g., altered or damaged such that it can no longer perform its intended purpose, in order to separate one from the other. Furthermore, in some versions, a chamber of a flow path is within, e.g., enclosed within, a limb enclosure unit, or a portion thereof.

Embodiments of the subject devices include devices in which a chamber of a liquid flow path is associated with, e.g., adjacent to, attached to and/or contacting, a portion e.g., a majority portion or entire portion, of a surface area of an enclosure unit wall that is opposite a limb associating wall of the enclosure unit. Various embodiments of the subject devices also include devices in which a chamber of a liquid flow path is associated with, e.g., adjacent to, attached to and/or contacting, a portion e.g., a majority portion or entire portion, of a surface area of a surface which is opposite a limb associating surface.

Also, as noted above, an enclosure unit wall that is opposite a limb associating wall of the enclosure unit may have a surface area which may be associated with, for example, a chamber of a liquid flow path. In such an embodiment, a chamber of a liquid flow path may be associated with, e.g., attached to and/or adjacent to and/or contacting, a portion of the surface area of the wall, such as a majority or minority portion or a portion which is 10% or more, or 50% or more, or 90% or more of the surface area of the wall. Also, in various embodiments, heat may be transferred from a warm liquid, e.g., a warm liquid within a chamber of a liquid flow path, to a limb via one or more walls of an enclosure unit.

In some embodiments, the liquid flow path includes a propulsion element, such as a pump, for inducing movement of liquid, e.g., creating a flow of liquid, through the liquid flow path. In certain variations, a pump causes liquid to move unidirectionally or bidirectionally through a flow path, or one or more components thereof. In some instances, a pump is electrically powered or manually powered, and in some embodiments may be a syringe. In certain aspects, a pump is an automatic pump and/or is operably connected to a power source, e.g., an electrical power source.

In particular aspects, liquid flow paths include one or more valves for controlling the movement of liquid through the components. In some embodiments, the valves are controllable (e.g., configured to be opened and/or closed in reaction to a designated signal or action). Where desired, each valve is individually controllable (e.g., a valve may be opened and or closed while other valves are not). In certain embodiments, the one or more valves include electrical components and may be configured to receive an electronic signal from a controller, e.g., a computer, operably connected thereto.

In various aspects of the subject disclosure, the liquid flow path, or components thereof, such as a chamber, includes one or more turbulence inducer. A turbulence inducer may be one or more structures, within the liquid flow path or components thereof, e.g., a chamber, which are configured to induce a turbulent flow of a liquid, e.g., a warm liquid, therethrough. A turbulence inducer may include one or more projections, such as inwardly-directed projections or projections connecting a first side of a chamber with a second opposite side of a chamber. Such projections may extend, e.g., extend inwardly, from an inner surface of a chamber of a liquid flow path. Turbulence inducers, according to the subject embodiments, may also include one or more material, such as a screen, filter, or mesh, positioned within the flow path. Turbulence inducers may also be transverse, e.g., perpendicular, to a flow of liquid within the flow path. A turbulence inducer may be configured to interrupt a direct flow of liquid, e.g., a flow of liquid substantially in a single direction, such as a single direction at a point within the flow path, and/or may create an indirect flow of liquid within a liquid flow path. Turbulence inducers may form a matrix, e.g., a dot-matrix, pattern on an inner surface of a chamber. Turbulence inducers also may include one or more walls, such as walls extending from an inner surface of a chamber of a liquid flow path, which direct a liquid flowing therethrough in a serpentine pattern.

Also, in some embodiments, the shape of a chamber itself induces turbulence. For example, a chamber may be shaped as a conduit, e.g., a tube, such as a coiled tube, which directs fluid in a turbulent flow. Such a chamber may direct liquid flowing therethrough in a serpentine pattern, such as in a serpentine flow path. In such an embodiment, the chamber may direct liquid flowing therethrough around a limb contained within the enclosure unit. In other such embodiments, the chamber may not direct liquid flowing therethrough around a limb contained within the enclosure unit but instead may direct liquid flowing therethrough adjacent to such a limb.

A noted above, in some versions, a liquid flow path is configured to bring, e.g., flow by pumping, a liquid, e.g., a warm liquid, in heat transfer relationship with a limb of a non-human mammal, or a portion thereof, enclosed in the enclosure unit. Fluids, according to the subject disclosure may include one or more substances in a liquid phase and/or gas phase. Such fluids may include water, e.g., water having a concentration of salt, such as sodium-chloride, dissolved therein. Also, as used here in a “warm liquid” refers to a liquid having a higher temperature than the body temperature of a non-human mammal, such as a non-human mammal with hypothermia. A warm liquid may also have a higher temperature than the temperature of the environment immediately surrounding the liquid. As such, a warm liquid may also have a higher temperature than room temperature, e.g., 20° C., 25° C., or 30° C., which is the temperature of the room in which the liquid is in. Temperature of a warm liquid may be 1° C., 5° C., 10° C., or 15° C. or more than room temperature. Temperature of a warm liquid may be 1° C., 5° C., 10° C., or 15° C. or more greater than the body temperature of a non-human mammal, such as a non-human mammal with hypothermia. Temperature of a warm liquid may range, for example, from 20° C. to 50° C., such as from 25° C. to 45° C., such as from 30° C. to 40° C., each inclusive. Temperature of a warm liquid may range, for example, from 35° C. to 50° C., such as from 40° C. to 50° C., such as from 45° C. to 50° C., or from 40° C. to 45° C., each inclusive. Temperature of a warm liquid may be 35° C. or more, such as 40° C. or more, such as 45° C. or more. Temperature of a warm liquid may be 35° C. or less, such as 40° C. or less, such as 45° C. or less, each inclusive. Temperature of a warm liquid may be 40° C., 41° C., 42° C., 43° C., 44° C., or 45° C. By “inclusive”, as used herein in association with ranges means that the range includes both of the provided endpoints. All ranges provided herein are inclusive, regardless of whether the term is provided in association with a particular range or unless indicated otherwise.

As used herein, the phrase “heat transfer relationship” refers to an association, such as a spatial positioning, of two or more aspects, e.g., a liquid and a limb, in which energy e.g., thermal energy, e.g., heat, may be exchanged between the two or more aspects. For example, heat may be transferred from a liquid in a heat transfer relationship to a limb where there is a heat transfer relationship between the liquid and the limb. Creating a heat transfer relationship, in some versions, may include elevating the body temperature of a non-human mammal, via its limb, by 1° C., 5° C., 10° C., or 15° C. or greater. Heat may also be transferred to a liquid in a heat transfer relationship from a limb where there is a heat transfer relationship between the liquid and the limb. Creating a heat transfer relationship may include positioning a liquid or a flow thereof adjacent to and/or contacting a limb. Creating a heat transfer relationship may also include positioning a liquid, or a flow thereof, adjacent to and/or contacting a first surface of a limb enclosure unit or a portion thereof, such as a heat transfer layer, and/or positioning a limb or a portion thereof, adjacent to and/or contacting a second surface, e.g., a second surface opposite the first surface, of a limb enclosure unit or a portion thereof, such as a heat transfer layer. Creating a heat transfer relationship may also include exchanging heat between a first aspect, e.g., a liquid, and a second aspect, e.g., a limb, via a limb enclosure unit or a portion thereof, such as a heat transfer layer.

According to some embodiments, liquid flow paths may include one or more heating elements for heating a liquid within the flow path. Examples of such heating elements include thermoelectric heating elements, e.g., that may include a resistive conductors, Peltier devices, or other elements that generate heat. Examples of thermoelectric heating elements of interest include, but are not limited to, those described in U.S. Pat. Nos. 8,567,861; 7,663,378; 7,500,536; 7,202,444; 6,150,642; 5,894,207; 5,111,025; 4,825,048 and 4,700,046; the disclosure of the heating elements thereof being incorporated herein by reference.

In some embodiments, one or more elements of the subject devices, e.g., a propulsion element, e.g., a pump, and/or a liquid heating element, e.g., heater, of a liquid flow path, are operatively connected (e.g., electrically coupled) to a power source. A power source may, in some aspects, be a battery, e.g., a portable and/or self-contained battery, an outlet, or another source of electrical power. In some aspects, a power source may include one or more electrical cords, e.g., cords configured to operatively connect a device to an outlet. Cords of power sources may be configured to removably connect to a pump and/or an outlet.

Devices as described herein and portions thereof, e.g., enclosure units and/or liquid flow paths, may be fabricated from any convenient material or combination of materials. Materials of interest include, but are not limited to: polymeric materials, e.g., plastics and/or rubbers, such as polytetrafluoroethene or polytetrafluoroethylene (PFTE), including expanded polytetrafluoroethylene (e-PFTE), polyester (Dacron™), nylon, polypropylene, polyethylene, high-density polyethylene (HDPE), polyurethane, etc., metals, and metal alloys, e.g., titanium, chromium, stainless steel, etc., and the like.

Materials of which enclosure units and/or liquid flow paths or portions thereof may be composed include materials which have the requisite strength for the disclosed devices and methods to effectively be employed. For example, materials of enclosure units and/or liquid flow paths may be composed include materials which are resistant to degrading and/or tearing and/or breaking when such elements are repeatedly employed according to the subject methods. Materials of which enclosure units and/or liquid flow paths or portions thereof may be composed also include materials which are sterilizable and/or re-sterilizable. Materials of which enclosure units and/or liquid flow paths are composed, in various embodiments, also include biodegradable and/or organic materials.

### Methods

The subject disclosure includes methods of ameliorating hypothermia or risk of occurrence thereof in a mammal, such as a non-human mammal, during a surgical procedure. In some versions, the methods include positioning a limb of the mammal, or a portion thereof, in an enclosure unit of a hypothermia amelioration device. Positioning a limb of the mammal, or a portion thereof, in an enclosure unit my include inserting a limb or a portion of a limb, e.g., an end of a limb, into an enclosure unit. Such a limb may, in some aspects, then be secured inside the enclosure device, such as by tightening the device around the limb, for a period of time, e.g., for a duration of a surgical procedure. A limb, or a portion thereof, such as an end of a limb, may then be removed from an enclosure unit.

According to various aspects of the methods, an enclosure unit is configured to enclose at least a substantial portion, e.g., a majority portion, of a limb of a non-human mammal, e.g., a small, non-human mammal. Also, in various aspects, a device, and in some versions, an enclosure unit thereof, includes a liquid flow path associated with, e.g., integrated with and/or attached to one or more portions of, the enclosure unit. A liquid flow pay may, in some aspects, be configured to bring a warm liquid in heat transfer relationship with the limb of a non-human mammal present in, such as contained within, the enclosure unit.

Various aspects of the disclosed methods also include moving, e.g., propelling, such as by pumping, a liquid, e.g., a warm liquid, through the flow path in a manner sufficient to ameliorate hypothermia or the risk of occurrence thereof in the non-human animal. In some versions the flow path includes a fluidic loop and the methods include re-circulating the liquid through the fluidic loop. In some aspects, the flow path includes a liquid inlet, a liquid outlet and/or a chamber. In various embodiments, the chamber is in operative, e.g., fluidic, communication with the liquid inlet and/or the liquid outlet. In various embodiments, the methods include moving, e.g., propelling, such as by pumping, a liquid, e.g., a warm liquid, through one or more of the liquid inlet, liquid outlet and/or chamber. In some aspects, the methods include transferring energy, e.g., thermal energy, such as heat, from a liquid, such as a warm liquid, to a limb of a non-human mammal. The methods may also include elevating and/or maintaining the body temperature of a non-human mammal.

In some versions the flow path includes a liquid inlet and the methods include moving, e.g., pumping, a liquid into the liquid inlet and/or into a chamber of the flow path via the liquid inlet. In some versions the flow path includes a liquid outlet and the methods include moving, e.g., pumping, a liquid out of the liquid outlet and/or out of a chamber of the flow path via the liquid outlet.

The methods, according to some embodiments, include enclosing a portion, such as 10% or more, or 50% or more, or 90% or more of the surface area of a limb of a non-human mammal in an enclosure unit. In some embodiments of the methods, an enclosure unit is configured as a boot and the methods include inserting a limb into a boot and/or removing the limb from the boot. Also, according to some aspects, the methods include retaining and/or securing a limb inside an enclosure unit. Securing a limb may include tightening the enclosure unit or a portion thereof around a limb for a duration of time, such as for the duration of a surgical procedure. The enclosure unit of a device, in some aspects includes one or more limb securing element which may be used to secure a limb inside the enclosure unit. A limb securing element may include one or more strap, such as an adjustable strap. In some versions, a strap may be associated with an opening of an enclosure unit and/or may be configured for shrinking and/or enlarging the opening around a limb of a non-human mammal.

In some versions of the methods, the methods include inducing a turbulent flow of liquid within a chamber of a flow path. In some aspects, the chambers include one or more turbulence inducer and the methods include flowing a liquid against the one or more turbulence inducer to create a turbulent flow. Also, according to some aspects, a chamber of a device has a serpentine shape and the method includes directing a warm liquid in a serpentine flow path through the chamber. In some versions, the methods include directing a liquid around a limb of a non-human mammal or a portion thereof and/or adjacent to a limb of a non-human mammal.

According to embodiments of the methods, the methods include performing a surgical procedure on a non-human mammal. In some versions of the methods, a non-human mammal is sedated, such as sedated during a surgical procedure. As such, in some aspects, the methods include sedating a non-human mammal. Sedating a non-human mammal may include anesthetizing the non-human mammal using conventional anesthesia and anesthesiology procedures.

In some aspects, the methods include inserting a limb or a portion thereof of a non-human mammal having hypothermia or at risk for hypothermia into an enclosure unit of a device. The methods include operating the device so that liquid, e.g., warm liquid, circulates in a liquid flow path of the device and thereby transfers energy, e.g., thermal energy, such as heat, to the portion of the limb inserted into the enclosure unit. By adjusting, e.g., increasing and/or decreasing the temperature of the liquid in the flow path, the body temperature of the non-human mammal can be controlled, e.g., elevated, maintained and/or lowered. Controlling the body temperature of the non-human mammal may be applied to alleviate hypothermia in the mammal or a risk thereof. Alleviating hypothermia in the non-human mammal or a risk thereof may increase the lifespan of the non-human mammal and/or assist the mammal in being comfortable. As such, in some embodiments, the methods include increasing the lifespan of a non-human mammal and/or making a non-human mammal more comfortable.

Furthermore, in some embodiments of the methods, the methods include monitoring a non-human mammal's body temperature for a duration of time, such as a duration of time during a surgical procedure, using, for example, a thermometer. Also, in some versions, the methods include increasing and/or decreasing and/or maintaining a body temperature of a non-human mammal based on the mammal's body temperature, such as the body temperature indication provided by the thermometer.

As noted above, subject devices are simple to use. First, a small non-human mammal's limb is inserted through an opening and thereby placed inside a limb enclosure unit. The limb is then secured within the enclosing device using for example, one or more limb securing elements. A surgical procedure may then be performed on the non-human mammal. Simultaneously, a liquid, e.g., a warm liquid may be moved through the liquid flow path. Moving the liquid through the flow path may transfer energy, e.g., heat energy, to the portion of the limb of the non-human mammal contained within the enclosure unit. By transferring heat energy to the non-human mammal, the non-human mammal's body temperature can be controlled, e.g., elevated and/or maintained, to alleviate hypothermia, or risk thereof. The non-human mammal's body temperature can also be monitored using, for example, a thermometer, throughout the surgical procedure. Following the surgical procedure, the limb securing elements may be adjusted, e.g., loosened, and the limb removed from the enclosure unit.

### Systems and Kits

The embodiments disclosed herein also include systems, such as systems including one or more, e.g., two, three, four, or five, or more, devices, such a devices which are operatively connected to one another within the system. The subject systems may also include a source of a liquid, such as a temperature controlled liquid and/or a warm liquid.

Aspects of the subject systems include hypothermia amelioration devices and may include a limb enclosure unit and a liquid flow path associated with the enclosure unit. Such devices may be configured for ameliorating surgical procedure associated hypothermia or risk of occurrence thereof in a small non-human mammal. According to some aspects, a limb enclosure unit is configured to enclose at least a substantial portion of a limb of a small non-human mammal. Also, in some versions, a liquid flow path is configured to bring a warm liquid in heat transfer relationship with a limb of a small non-human mammal present in the enclosure unit.

As noted above, the subject systems may also include a source of a liquid, such as a temperature controlled liquid and/or a warm liquid. By “temperature controlled liquid” is a liquid having a temperature which is maintained, elevated and/or lowered and which may be circulated through one or more devices as described herein for ameliorating hyperthermia, such as surgical procedure associated hypothermia, or risk of occurrence thereof in a small non-human mammal. A source of a liquid, such as a temperature controlled liquid and/or a warm liquid, may include one or more container, e.g., a sealed container, for containing the liquid and/or one or more propulsion element, such as a pump, and/or one or more conduits, e.g., tubes, for directing the liquid to and/or from one or more device as described herein.

Also provided are kits that at least include the subject devices and which may be used according to the subject methods. The subject kits may include two or more, e.g., a plurality, three, four, five, ten, etc., devices for ameliorating hypothermia or risk thereof according to any of the embodiments described herein, or any combinations thereof. In addition, the kits may include any device or other element which may facilitate the operation of any aspect of the kits. For example, a kit may include one or more devices for ameliorating hypothermia and/or one or more elements of such a device. Kits may also include packaging, e.g., packaging for shipping the devices without breaking.

In certain embodiments, the kits which are disclosed herein include instructions, such as instructions for using devices. The instructions for using devices are, in some aspects, recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging etc.). In other embodiments, the instructions are present as an electronic storage data file present on a suitable computer readable storage medium, e.g., Portable Flash drive, CD-ROM, diskette, etc. The instructions may take any form, including complete instructions for how to use the devices or as a website address with which instructions posted on the world wide web may be accessed.

### Utility

As demonstrated above, the subject devices and methods are directed to ameliorating hypothermia, such as surgical procedure associated hypothermia, or risk of occurrence thereof in a non-human mammal, such as a dog or a cat. As such, the subject methods are suitable for use in a variety of different applications, where particular applications include addressing normal and abnormal physiological conditions, such as hypothermia or risk thereof, where body heat, e.g., core body heat, elevation and/or maintenance may be desirable. Particular applications in which the subject devise and methods find use include the alleviation or treatment of hypothermia during a surgical procedure. By “treatment” is meant at least an alleviation in one or more of the symptoms associated with the condition being treated, e.g. a reduction in discomfort, amelioration or elimination of symptoms, such as a low body temperature, etc.

In some aspects, a non-human mammal having hypothermia or at risk for hypothermia may one of its limbs, or a portion thereof, inserted into an enclosure unit of a device. The device may then be operated so that liquid, e.g., warm liquid, circulates in a liquid flow path of the device and thereby transfers energy, e.g., thermal energy, such as heat, to the portion of the limb inserted into the enclosure unit. By adjusting, e.g., increasing and/or decreasing the temperature of the liquid in the flow path, the body temperature of the non-human mammal can be controlled, e.g., elevated, maintained and/or lowered. Controlling the body temperature of the non-human mammal may be applied to alleviate hypothermia in the mammal or a risk thereof. Alleviating hypothermia in the non-human mammal or a risk thereof may increase the lifespan of the non-human mammal and/or assist the mammal in being comfortable. Alleviating hypothermia in the non-human mammal or a risk thereof during a surgical procedure may also increase the success rate of a surgical procedure performed on the mammal and/or reduce the health risk to the non-human mammal of the surgical procedure.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A hypothermia amelioration device for ameliorating surgical procedure associated hypothermia or risk of occurrence thereof in a small non-human mammal, the device comprising:
a limb enclosure unit configured to enclose at least a substantial portion of a limb of a small non-human mammal; and
a liquid flow path associated with the enclosure unit and configured to bring a warm liquid in heat transfer relationship with a limb of a small non-human mammal present in the enclosure unit.

2. The device according to claim 1, wherein the enclosure unit is configured to enclose 90% or more of the surface area of a limb of a small non-human mammal.

3. The device according to claim 1, where the enclosure unit is configured as a boot.

4. The device according to claim 1, wherein the enclosure unit further comprises a limb securing element.

5. The device according to claim 4, wherein the limb securing element comprises an adjustable strap associated with an opening of the enclosure unit.

6. The device according to claim 1, wherein the liquid flow path comprises a liquid inlet, a liquid outlet and a chamber in fluidic communication with the liquid inlet and liquid outlet.

7. The device according to claim 6, wherein the chamber is associated with a majority of the surface area of the enclosure unit wall that is opposite the limb associating wall of the enclosure unit.

8. The device according to claim 6, wherein the chamber further comprises a turbulence inducer.

9. The device according to claim 8, where the turbulence inducer comprises a projection extending from an inner surface of the chamber.

10. The device according to claim 6, wherein the chamber has a serpentine shape and directs the warm liquid in a serpentine flow path.

11. The device according to claim 1, wherein the small non-human mammal is a cat or dog.

12. A method of ameliorating hypothermia or risk of occurrence thereof in a small non-human mammal during a surgical procedure, the method comprising:
(a) positioning a limb of the mammal in an enclosure unit of a hypothermia amelioration device, wherein the enclosure unit is configured to enclose at least a substantial portion of a limb of the small non-human mammal and comprises a liquid flow path associated with the enclosure unit that is configured to bring a warm liquid in heat transfer relationship with the limb of a small non-human mammal present in the enclosure unit;
(b) moving a warm liquid through the flow path in a manner sufficient to ameliorate hypothermia or the risk of occurrence thereof in the small non-human mammal.

13. The method according to claim 12, wherein the enclosure unit is configured to enclose 90% or more of the surface area of a limb of a small non-human mammal.

14. The method according to claim 12, where the enclosure unit is configured as a boot.

15-16. (canceled)

17. The method according to claim 12, wherein the liquid flow path comprises a liquid inlet, a liquid outlet and a chamber in fluidic communication with the liquid inlet and liquid outlet.

18. The method according to claim 17, wherein the chamber is associated with a majority of the surface area of enclosure unit wall that is opposite the limb associating wall of the enclosure unit.

19-21. (canceled)

22. The method according to claim 12, wherein the small non-human mammal is a cat or dog.

23. The method according to claim 12, wherein the small non-human mammal is sedated.

24. The method according to claim 12, wherein the method further comprises performing a surgical procedure on the small non-human mammal.

25. A system comprising:
(a) a device according to claim 1; and
(b) a source of a temperature controlled liquid.

26-27. (canceled)
